(19) United States
(12) Patent Application Publication
(10) Pub. No.: US 2002/0039425 A1
(43) Pub. Date: Apr. 4, 2002

(54) Method and apparatus for removing noise from electronic signals

(76) Inventors: Gregory C. Burnett, Livermore, CA (US); Eric F. Breitfeller, San Francisco, CA (US)

Correspondence Address: WILSON SONSINI GOODRICH & ROSATI, 650 PAGE MILL ROAD, PALO ALTO, CA, 943041050

(21) Appl. No.: 09/905,361

(22) Filed: Jul. 12, 2001

Related U.S. Application Data
(60) Provisional application No. 60/219,297, filed on Jul. 19, 2000.

(51) Int. Cl. H03B 29/00; A61F 11/06; H04B 15/00

(52) U.S. Cl. 381/94.7; 381/71.1

(57) ABSTRACT

A method and system are provided for acoustic noise removal from human speech, wherein noise is removed without respect to noise type, amplitude, or orientation. The system includes microphones and a voice activity detection (VAD) data stream coupled among a processor. The microphones receive acoustic signals and the VAD produces a signal including a binary one when speech (voiced and unvoiced) is occurring and a binary zero in the absence of speech. The processor includes denoising algorithms that generate transfer functions. The transfer functions include a transfer functions generated in response to a determination that voicing information is absent from the received acoustic signal during a specified time period. The transfer functions also include transfer functions generated in response to a determination that voicing information is present in the acoustic signal during a specified time period. At least one denoised acoustic data stream is generated using the transfer functions.

### RELATED APPLICATIONS

This application claims the benefit of United States Provisional Patent Application No. 60/219,297, filed Jul. 19, 2000, incorporated herein by reference.

### FIELD OF THE INVENTION

The invention is in the field of mathematical methods and electronic systems for removing or suppressing undesired acoustical noise from acoustic transmissions or recordings.

### BACKGROUND

In a typical acoustic application, speech from a human user is recorded or stored and transmitted to a receiver in a different location. In the environment of the user, there may exist one or more noise sources that pollute the signal of interest (the user's speech) with unwanted acoustic noise. This makes it difficult or impossible for the receiver, whether human or machine, to understand the user's speech. This is especially problematic now with the proliferation of portable communication devices like cellular telephones and personal digital assistants. There are existing methods for suppressing these noise additions, but they either require far too much computing time or cumbersome hardware, distort the signal of interest too much, or lack in performance to be useful. Many of these methods are described in textbooks such as “Advanced Digital Signal Processing and Noise Reduction” by Vaseghi, ISBN 0-471-62692-9. Consequently, there is a need for noise removal and reduction methods that address the shortcomings of typical systems and offer new techniques for cleaning acoustic signals of interest without distortion.

### SUMMARY

A method and system are provided for acoustic noise removal from human speech, wherein the noise can be removed and the signal restored without respect to noise type, amplitude, or orientation. The system includes microphones and sensors coupled with a processor. The microphones receive acoustic signals including both noise and speech signals from human signal sources. The sensors yield a binary Voice Activity Detection (VAD) signal that provides a signal that is a binary “1” when speech (both voiced and unvoiced) is occurring and a binary “0” when no speech is occurring. The VAD signal can be obtained in numerous ways, for example, using acoustic gain, accelerometers, and radio frequency (RF) sensors.

The processor system and method includes denoising algorithms that calculate the transfer function among the noise sources and the microphones as well as the transfer function among the human user and the microphones. The transfer functions are used to remove noise from the received acoustic signal to produce at least one denoised acoustic data stream.

### BRIEF DESCRIPTION OF THE DRAWINGS

FIG. 1 is a block diagram of a denoising system of an embodiment.

FIG. 2 is a block diagram of a noise removal algorithm of an embodiment, assuming a single noise source and a direct path to the microphones.

FIG. 3 is a block diagram of a front end of a noise removal algorithm of an embodiment, generalized to n distinct noise sources (these noise sources may be reflections or echoes of one another).

FIG. 4 is a block diagram of a front end of a noise removal algorithm of an embodiment in the most general case where there are n distinct noise sources and signal reflections.

FIG. 5 is a flow diagram of a denoising method of an embodiment.

FIG. 6 shows results of a noise suppression algorithm of an embodiment for an American English female speaker in the presence of airport terminal noise that includes many other human speakers and public announcements.

### DETAILED DESCRIPTION

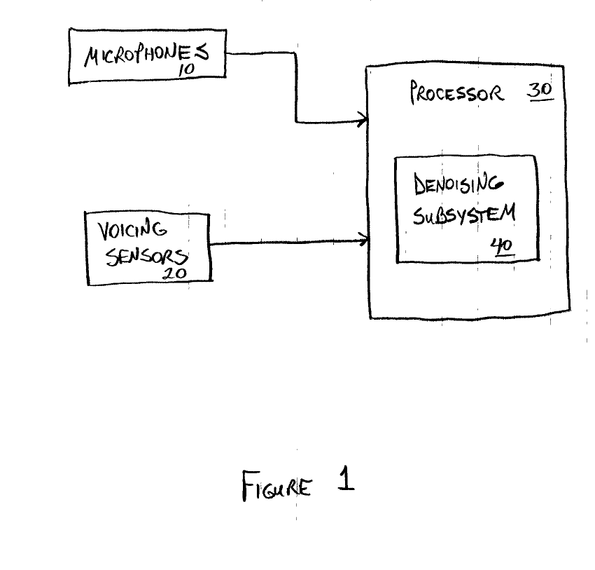

FIG. 1 is a block diagram of a denoising system of an embodiment that uses knowledge of when speech is occurring derived from physiological information on voicing activity. The system includes microphones 10 and sensors 20 that provide signals to at least one processor 30. The processor includes a denoising subsystem or algorithm.

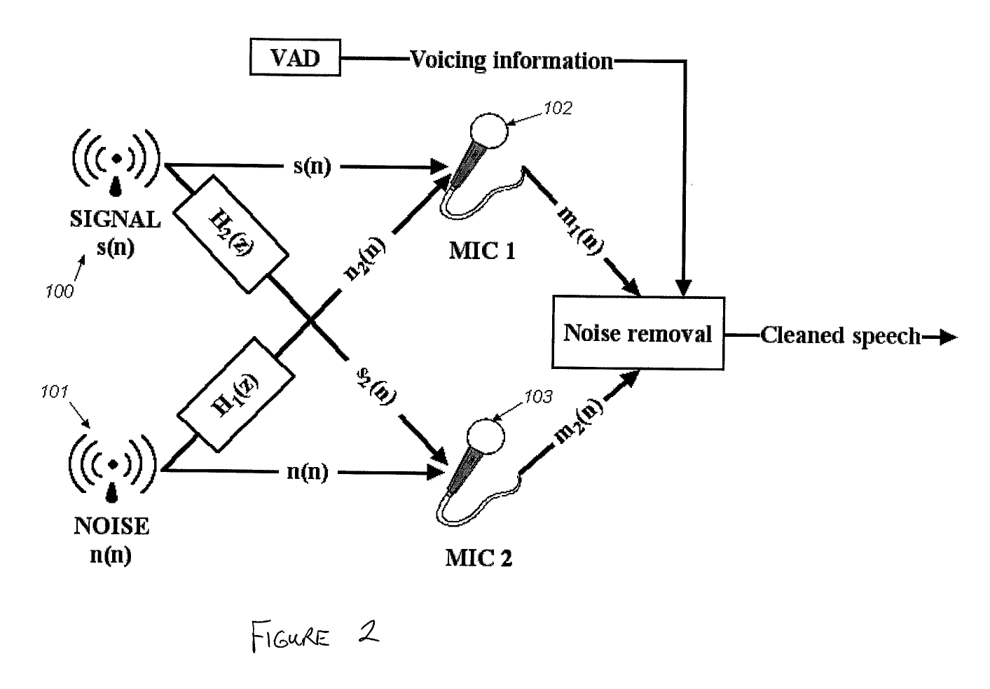

FIG. 2 is a block diagram of a noise removal system/algorithm of an embodiment, assuming a single noise source and a direct path to the microphones. The noise removal system diagram includes a graphic description of the process of an embodiment, with a single signal source (100) and a single noise source (101). This algorithm uses two microphones, a “signal” microphone (MIC 1, 102) and a “noise” microphone (MIC 2, 103), but is not so limited. MIC 1 is assumed to capture mostly signal with some noise, while MIC 2 captures mostly noise with some signal. This is the common configuration with conventional advanced acoustic systems. The data from the signal to MIC 1 is denoted by s(n), from the signal to MIC 2 by s₂(n), from the noise to MIC 2 by n(n), and from the noise to MIC 1 by n₂(n). Similarly, the data from MIC 1 is denoted by m₁(n), and the data from MIC 2 m₂(n), where s(n) denotes a discrete sample of the analog signal from the source.

The transfer functions from the signal to MIC 1 and from the noise to MIC 2 are assumed to be unity, but the transfer function from the signal to MIC 2 is denoted by H₂(z) and from the noise to MIC 1 by H₁(z). The assumption of unity transfer functions does not inhibit the generality of this algorithm, as the actual relations between the signal, noise, and microphones are simply ratios and the ratios are redefined in this manner for simplicity.

In conventional noise removal systems, the information from MIC 2 is used to attempt to remove noise from MIC 1. However, an unspoken assumption is that the Voice Activity Detection (VAD) is never perfect, and thus the denoising must be performed cautiously, so as not to remove too much of the signal along with the noise. However, if the VAD is assumed to be perfect and is equal to zero when there is no speech being produced by the user, and one when speech is produced, a substantial improvement in the noise removal can be made.

In analyzing the single noise source and direct path to the microphones, with reference to FIG. 2, the acoustic information coming into MIC 1 is denoted by m₁(n). The information coming into MIC 2 is similarly labeled m₂(n). In the z (digital frequency) domain, these are represented as M₁(z) and M₂(z). Then

M₁(z)=S(z)+N₂(z)

M₂(z)=N(z)+S₂(z)

with

N₂(z)=N(z)H₁(z)

S₂(z)=S(z)H₂(z)

so that

M₁(z)=S(z)+N(z)H₁(z)

M₂(z)=N(z)+S(z)H₂(z) Eq. 1

This is the general case for all two microphone systems. In a practical system there is always going to be some leakage of noise into MIC 1, and some leakage of signal into MIC 2. Equation 1 has four unknowns and only two known relationships and therefore cannot be solved explicitly.

However, there is another way to solve for some of the unknowns in Equation 1. The analysis starts with an examination of the case where the signal is not being generated, that is, where the VAD signal equals zero and speech is not being produced. In this case, s(n)=S(z)=0, and Equation 1 reduces to

M₁ₙ(z)=N(z)H₁(z)

M₂ₙ(z)=N(z)

where the n subscript on the M variables indicate that only noise is being received. This leads to

$$\begin{matrix}{M}_{1 n} ( z ) = {M}_{2 n} ( z ) {H}_{1} ( z ) & Eq . 2 \\ {H}_{1} ( z ) = \frac{{M}_{1 n} ( z )}{{M}_{2 n} ( z )} . & \end{matrix}$$

H₁(z) can be calculated using any of the available system identification algorithms and the microphone outputs when the system is certain that only noise is being received. The calculation can be done adaptively, so that the system can react to changes in the noise.

A solution is now available for one of the unknowns in Equation 1. Another unknown, H₂(z), can be determined by using the instances where the VAD equals one and speech is being produced. When this is occurring, but the recent (perhaps less than 1 second) history of the microphones indicate low levels of noise, it can be assumed that n(s)=N(z)˜0. Then Equation 1 reduces to

M₁ₛ(z)=S(z)

M₂ₛ(z)=S(z)H₂(z)

which in turn leads to

$${M}_{2 s} ( z ) = {M}_{1 s} ( z ) {H}_{2} ( z )$$

$${H}_{2} ( z ) = \frac{{M}_{2 s} ( z )}{{M}_{1 s} ( z )}$$

which is the inverse of the H₁(z) calculation. However, it is noted that different inputs are being used—now only the signal is occurring whereas before only the noise was occurring. While calculating H₂(z), the values calculated for H₁(z) are held constant and vice versa. Thus, it is assumed that H₁(z) and H₂(z) do not change substantially while the other is being calculated.

After calculating H₁(z) and H₂(z), they are used to remove the noise from the signal. If Equation 1 is rewritten as

S(z)=M₁(z)−N(z)H₁(z)

N(z)=M₂(z)−S(z)H₂(z)

S(z)=M₁(z)−[M₂(z)−S(z)H₂(z)]H₁(z)′

S(z)[1−H₂(z)H₁(z)]=M₁(z)−M₂(z)H₁(z)

then N(z) may be substituted as shown to solve for S(z) as:

$$S ( z ) = \frac{{M}_{1} ( z ) - {M}_{2} ( z ) {H}_{1} ( z )}{1 - {H}_{2} ( z ) {H}_{1} ( z )} \qquad Eq . 3$$

If the transfer functions H₁(z) and H₂(z) can be described with sufficient accuracy, then the noise can be completely removed and the original signal recovered. This remains true without respect to the amplitude or spectral characteristics of the noise. The only assumptions made are a perfect VAD, sufficiently accurate H₁(z) and H₂(z), and that H₁(z) and H₂(z) do not change substantially when the other is being calculated. In practice these assumptions have proven reasonable.

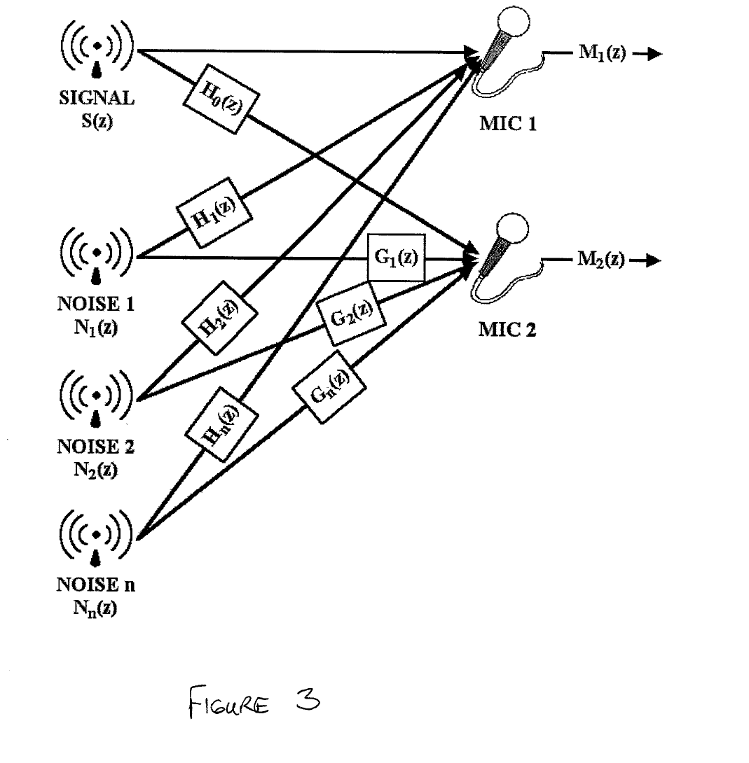

The noise removal algorithm described herein is easily generalized to include any number of noise sources. FIG. 3 is a block diagram of a front end of a noise removal algorithm of an embodiment, generalized to n distinct noise sources. These distinct noise sources may be reflections or echoes of one another, but are not so limited. There are several noise sources shown, each with a transfer function, or path, to each microphone. The previously named path H₂ has been relabeled as H₀, so that labeling noise source 2's path to MIC 1 is more convenient. The outputs of each microphone, when transformed to the z domain, are:

M₁(z)=S(z)+N₁(z)H₁(z)+N₂(z)H₂(z)+. . . Nₙ(z)Hₙ(z)

M₂(z)=S(z)H₀(z)+N₁(z)G₁(z)+N₂(z)G₂(z)+. . . Nₙ(z)Gₙ(z) Eq. 4

When there is no signal (VAD=0), then (suppressing the z's for clarity)

M₁ₙ=N₁H₁+N₂H₂+. . . NₙHₙ

M₂ₙ=N₁G₁+N₂G₂+. . . NₙGₙ Eq. 5

A new transfer function can now be defined, analogous to H₁(z) above:

$${\tilde{H}}_{1} = \frac{{M}_{1 n}}{{M}_{2 n}} = \frac{{N}_{1} {H}_{1} + {N}_{2} {H}_{2} + … {N}_{n} {H}_{n}}{{N}_{1} {G}_{1} + {N}_{2} {G}_{2} + … {N}_{n} {G}_{n}} \qquad Eq . 6$$

Thus \tilde{H}₁ depends only on the noise sources and their respective transfer functions and can be calculated any time there is no signal being transmitted. Once again, the n subscripts on the microphone inputs denote only that noise is being detected, while an s subscript denotes that only signal is being received by the microphones.

Examining Equation 4 while assuming that there is no noise produces

M₁ₛ=S

M₂ₛ=SH₀

Thus H₀ can be solved for as before, using any available transfer function calculating algorithm. Mathematically

$${H}_{0} = \frac{{M}_{2 s}}{{M}_{1 s}}$$

Rewriting Equation 4, using \tilde{H}₁ defined in Equation 6, provides,

$${\tilde{H}}_{1} = \frac{{M}_{1} - S}{{M}_{2} - {SH}_{0}} \qquad Eq . 7$$

Solving for S yields,

$$S = \frac{{M}_{1} - {M}_{2} {\tilde{H}}_{1}}{1 - {H}_{0} {\tilde{H}}_{1}} \qquad Eq . 8$$

which is the same as Equation 3, with H₀ taking the place of H₂, and \tilde{H}₁ taking the place of H₁. Thus the noise removal algorithm still is mathematically valid for any number of noise sources, including multiple echoes of noise sources. Again, if H₀ and \tilde{H}₁ can be estimated to a high enough accuracy, and the above assumption of only one path from the signal to the microphones holds, the noise may be removed completely.

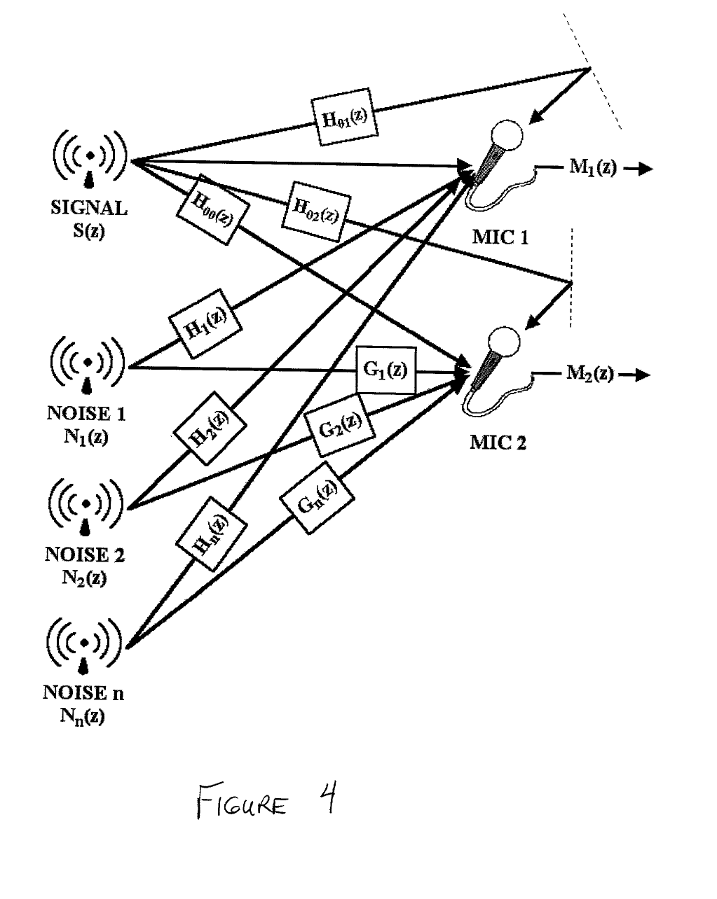

The most general case involves multiple noise sources and multiple signal sources. FIG. 4 is a block diagram of a front end of a noise removal algorithm of an embodiment in the most general case where there are n distinct noise sources and signal reflections. Here, reflections of the signal enter both microphones. This is the most general case, as reflections of the noise source into the microphones can be modeled accurately as simple additional noise sources. For clarity, the direct path from the signal to MIC 2 has changed from H₀(z) to H₀₀(z), and the reflected paths to Microphones 1 and 2 are denoted by H₀₁(z) and H₀₂(z), respectively.

The input into the microphones now becomes

M₁(z)=S(z)+S(z)H₀₁(z)+N₁(z)H₁(z)+N₂(z)H₂(z)+. . . Nₙ(z)Hₙ(z)

M₂(z)=S(z)H₀₀(z)+S(z)H₀₂(z)+N₁(z)G₁(z)+N₂(z)G₂(z)+. . . Nₙ(z)Gₙ(z) Eq. 9

When the VAD=0, the inputs become (suppressing the z's again)

M₁ₙ=N₁H₁+N₂H₂+. . . NₙHₙ

M₂ₙ=N₁G₁+N₂G₂+. . . NₙGₙ

which is the same as Equation 5. Thus, the calculation of \tilde{H}₁ in Equation 6 is unchanged, as expected. In examining the situation where there is no noise, Equation 9 reduces to

M₁ₛ=S+SH₀₁

M₂ₛ=SH₀₀+SH₀₂

This leads to the definition of \tilde{H}₂:

$${\tilde{H}}_{2} = \frac{{M}_{2 s}}{{M}_{1 s}} = \frac{{H}_{00} + {H}_{02}}{1 + {H}_{01}} \qquad Eq . 10$$

Rewriting Equation 9 again using the definition for \tilde{H}₁ (as in Equation 7) provides

$${\tilde{H}}_{1} = \frac{{M}_{1} - S ( 1 + {H}_{01} )}{{M}_{2} - S ( {H}_{00} + {H}_{02} )} \qquad Eq . 11$$

Some algebraic manipulation yields

$$S ( 1 + {H}_{01} - {\tilde{H}}_{1} ( {H}_{00} + {H}_{02} ) ) = {M}_{1} - {M}_{2} {\tilde{H}}_{1}$$

$$S ( 1 + {H}_{01} ) [ 1 - {\tilde{H}}_{1} \frac{( {H}_{00} + {H}_{02} )}{( 1 + {H}_{01} )} ] = {M}_{1} - {M}_{2} {\tilde{H}}_{1}$$

$$S ( 1 + {H}_{01} ) [ 1 - {\tilde{H}}_{1} {\tilde{H}}_{2} ] = {M}_{1} - {M}_{2} {\tilde{H}}_{1}$$

and finally

$$S ( 1 + {H}_{01} ) = \frac{{M}_{1} - {M}_{2} {\tilde{H}}_{1}}{1 - {\tilde{H}}_{1} {\tilde{H}}_{2}} \qquad Eq . 12$$

Equation 12 is the same as equation 8, with the replacement of H₀ by \tilde{H}₂, and the addition of the (1+H₀₁) factor on the left side. This extra factor means that S cannot be solved for directly in this situation, but a solution can be generated for the signal plus the addition of all of its echoes. This is not such a bad situation, as there are many conventional methods for dealing with echo suppression, and even if the echoes are not suppressed, it is unlikely that they will affect the comprehensibility of the speech to any meaningful extent. The more complex calculation of \tilde{H}₂ is needed to account for the signal echoes in Microphone 2, which act as noise sources.

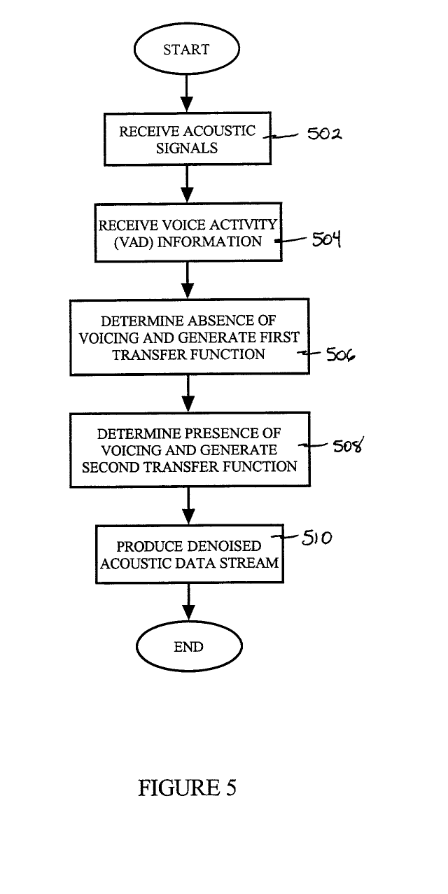

FIG. 5 is a flow diagram of a denoising method of an embodiment. In operation, the acoustic signals are received 502. Further, physiological information associated with human voicing activity is received 504. A first transfer function representative of the acoustic signal is calculated upon determining that voicing information is absent from the acoustic signal for at least one specified period of time 506. A second transfer function representative of the acoustic signal is calculated upon determining that voicing information is present in the acoustic signal for at least one specified period of time 508. Noise is removed from the acoustic signal using at least one combination of the first transfer function and the second transfer function, producing denoised acoustic data streams 510.

An algorithm for noise removal, or denoising algorithm, is described herein, from the simplest case of a single noise source with a direct path to multiple noise sources with reflections and echoes. The algorithm has been shown herein to be viable under any environmental conditions. The type and amount of noise are inconsequential if a good estimate has been made of \tilde{H}₁ and \tilde{H}₂, and if they do not change substantially while the other is calculated. If the user environment is such that echoes are present, they can be compensated for if coming from a noise source. If signal echoes are also present, they will affect the cleaned signal, but the effect should be negligible in most environments.

In operation, the algorithm of an embodiment has shown excellent results in dealing with a variety of noise types, amplitudes, and orientations. However, there are always approximations and adjustments that have to be made when moving from mathematical concepts to engineering applications. One assumption is made in Equation 3, where H₂(z) is assumed small and therefore H₂(z)H₁(z)≈0, so that Equation 3 reduces to

S(z)≈M₁(z)−M₂(z)H₁(z).

This means that only H₁(z) has to be calculated, speeding up the process and reducing the number of computations required considerably. With the proper selection of microphones, this approximation is easily realized.

Another approximation involves the filter used in an embodiment. The actual H₁(z) will undoubtedly have both poles and zeros, but for stability and simplicity an all-zero Finite Impulse Response (FIR) filter is used. With enough taps (around 60) the approximation to the actual H₁(z) is very good.

Regarding subband selection, the wider the range of frequencies over which a transfer function must be calculated, the more difficult it is to calculate it accurately. Therefore the acoustic data was divided into 16 subbands, with the lowest frequency at 50 Hz and the highest at 3700. The denoising algorithm was then applied to each subband in turn, and the 16 denoised data streams were recombined to yield the denoised acoustic data. This works very well, but any combinations of subbands (i.e. 4, 6, 8, 32, equally spaced, perceptually spaced, etc.) can be used and has been found to work as well.

The amplitude of the noise was constrained in an embodiment so that the microphones used did not saturate (i.e. operate outside a linear response region). It is important that the microphones operate linearly to ensure the best performance. Even with this restriction, very high signal-to-noise ratios (SNR) can be tested (down to about −10 dB).

The calculation of H₁(z) was accomplished every 10 milliseconds using the Least-Mean Squares (LMS) method, a common adaptive transfer function. An explanation may be found in “Adaptive Signal Processing” (1985), by Widrow and Stearns, published by Prentice-Hall, ISBN 0-13-004029-0.

The VAD for an embodiment was derived from a radio frequency sensor and the two microphones, yielding very high accuracy (>99%) for both voiced and unvoiced speech. The VAD of an embodiment uses a radio frequency (RF) interferometer to detect tissue motion associated with human speech production, but is not so limited. It is therefore completely acoustic-noise free, and is able to function in any acoustic noise environment. A simple energy measurement can be used to determine if voiced speech is occurring. Unvoiced speech can be determined using conventional frequency-based methods, by proximity to voiced sections, or through a combination of the above. Since there is much less energy in unvoiced speech, its activation accuracy is not as critical as voiced speech.

With voiced and unvoiced speech detected reliably, the algorithm of an embodiment can be implemented. Once again, it is useful to repeat that the noise removal algorithm does not depend on how the VAD is obtained, only that it is accurate, especially for voiced speech. If speech is not detected and training occurs on the speech, the subsequent denoised acoustic data can be distorted.

Data was collected in four channels, one for MIC 1, one for MIC 2, and two for the radio frequency sensor that detected the tissue motions associated with voiced speech. The data were sampled simultaneously at 40 kHz, then digitally filtered and decimated down to 8 kHz. The high sampling rate was used to reduce any aliasing that might result from the analog to digital process. A four-channel National Instruments A/D board was used along with Labview to capture and store the data. The data was then read into a C program and denoised 10 milliseconds at a time.

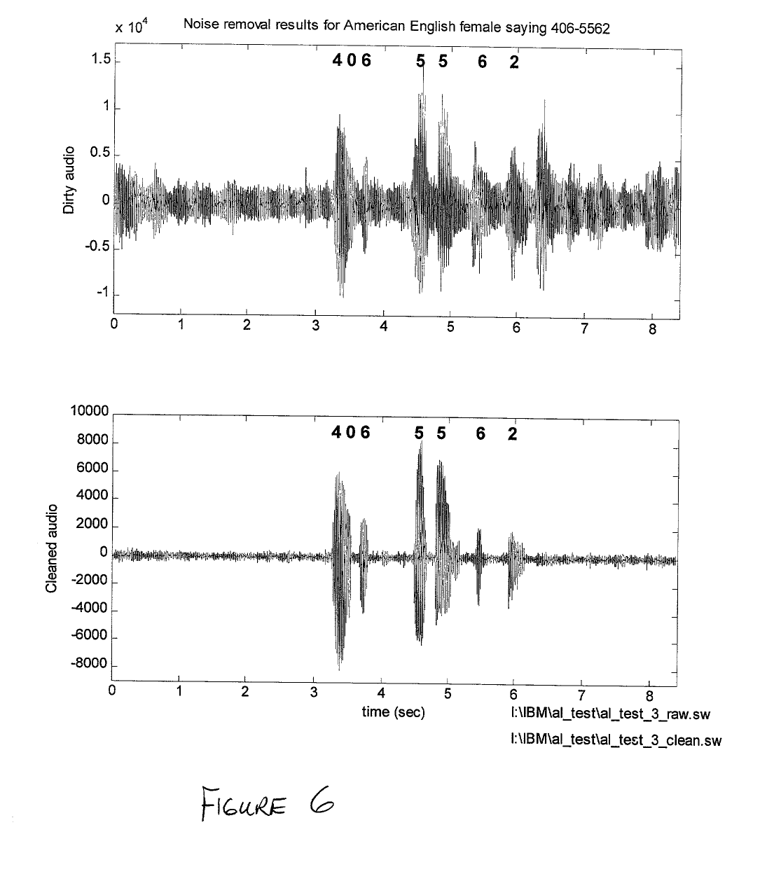

FIG. 6 shows results of a noise suppression algorithm of an embodiment for an American English speaking female in the presence of airport terminal noise that includes many other human speakers and public announcements. The speaker is uttering the numbers 406-5562 in the midst of moderate airport terminal noise. The dirty acoustic data was denoised 10 milliseconds at a time, and before denoising the 10 milliseconds of data were prefiltered from 50 to 3700 Hz. A reduction in the noise of approximately 17 dB is evident. No post filtering was done on this sample; thus, all of the noise reduction realized is due to the algorithm of an embodiment. It is clear that the algorithm adjusts to the noise instantly, and is capable of removing the very difficult noise of other human speakers. Many different types of noise have all been tested with similar results, including street noise, helicopters, music, and sine waves, to name a few. Also, the orientation of the noise can be varied substantially without significantly changing the noise suppression performance. Finally, the distortion of the cleaned speech is very low, ensuring good performance for speech recognition engines and human receivers alike.

The noise removal algorithm of an embodiment has been shown to be viable under any environmental conditions. The type and amount of noise are inconsequential if a good estimate has been made of \tilde{H}₁ and \tilde{H}₂. If the user environment is such that echoes are present, they can be compensated for if coming from a noise source. If signal echoes are also present, they will affect the cleaned signal, but the effect should be negligible in most environments.

Various embodiments are described herein with reference to the figures, but the detailed description and the figures are not intended to be limiting. Various combinations of the elements described have not been shown, but are within the scope of the invention which is defined by the following claims.

What is claimed is:

1. A method for removing noise from acoustic signals, comprising:
receiving a plurality of acoustic signals;
receiving physiological information associated with human voicing activity;
generating at least one first transfer function representative of the plurality of acoustic signals upon determining that voicing information is absent from the plurality of acoustic signals for at least one specified period of time;
generating at least one second transfer function representative of the plurality of acoustic signals upon determining that voicing information is present in the plurality of acoustic signals for the at least one specified period of time;
removing noise from the plurality of acoustic signals using at least one combination of the at least one first transfer function and the at least one second transfer function to produce at least one denoised acoustic data stream.

2. The method of claim 1, wherein the plurality of acoustic signals include at least one reflection of at least one associated noise source signal and at least one reflection of at least one acoustic source signal.

3. The method of claim 1, wherein receiving physiological information comprises receiving physiological data associated with human voicing using at least one detector selected from a group consisting of radio frequency devices, electroglottographs, ultrasound devices, acoustic throat microphones, and airflow detectors.

4. The method of claim 1, wherein receiving the plurality of acoustic signals includes receiving using a plurality of independently located microphones.

5. The method of claim 1, wherein removing noise further includes generating at least one third transfer function using the at least one first transfer function and the at least one second transfer function.

6. The method of claim 1, wherein generating the at least one first transfer function comprises recalculating the at least one first transfer function during at least one prespecified interval.

7. The method of claim 1, wherein generating the at least one second transfer function comprises recalculating the at least one second transfer function during at least one prespecified interval.

8. The method of claim 1, wherein generating the at least one first transfer function and the at least one second transfer function comprises use of at least one technique selected from a group consisting of adaptive techniques and recursive techniques.

9. A method for removing noise from electronic signals, comprising:
detecting an absence of voiced information during at least one period;
receiving at least one noise source signal during the at least one period;
generating at least one transfer function representative of the at least one noise source signal;
receiving at least one composite signal comprising acoustic and noise signals; and
removing the noise signal from the at least one composite signal using the at least one transfer function to produce at least one denoised acoustic data stream.

10. The method of claim 9, wherein the at least one noise source signal includes at least one reflection of at least one associated noise source signal.

11. The method of claim 9, wherein the at least one composite signal includes at least one reflection of at least one associated composite signal.

12. The method of claim 9, wherein detecting comprises collecting physiological data associated with human voicing using at least one detector selected from a group consisting of radio frequency devices, electroglottographs, ultrasound devices, acoustic throat microphones, and airflow detectors.

13. The method of claim 9, wherein receiving includes receiving the at least one noise source signal using at least one microphone.

14. The method of claim 13, wherein the at least one microphone includes a plurality of independently located microphones.

15. The method of claim 9, wherein removing the noise signal from the at least one composite signal using the at least one transfer function includes generating at least one other transfer function using the at least one transfer function.

16. The method of claim 9, wherein generating at least one transfer function comprises recalculating the at least one transfer function during at least one prespecified interval.

17. The method of claim 9, wherein generating the at least one transfer function comprises calculating the at least one transfer function using at least one technique selected from a group consisting of adaptive techniques and recursive techniques.

18. A method for removing noise from electronic signals, comprising:
determining at least one unvoicing period during which voiced information is absent;
receiving at least one noise signal input during the at least one unvoicing period and generating at least one unvoicing transfer function representative of the at least one noise signal;
determining at least one voicing period during which voiced information is present;
receiving at least one acoustic signal input from at least one signal sensing device during the at least one voicing period and generating at least one voicing transfer function representative of the at least one acoustic signal;
receiving at least one composite signal comprising acoustic and noise signals; and
removing the noise signal from the at least one composite signal using at least one combination of the at least one unvoicing transfer function and the at least one voicing transfer function to produce at least one denoised acoustic data stream.

19. A system for removing noise from acoustic signals, comprising:
at least one receiver that receives at least one acoustic signal;
at least one sensor that receives physiological information associated with human voicing activity;
at least one processor coupled among the at least one receiver and the at least one sensor that generates a plurality of transfer functions, wherein at least one first transfer function representative of the at least one acoustic signal is generated in response to a determination that voicing information is absent from the at least one acoustic signal for at least one specified period of time, wherein at least one second transfer function representative of the at least one acoustic signal is generated in response to a determination that voicing information is present in the at least one acoustic signal for at least one specified period of time, wherein noise is removed from the at least one acoustic signal using at least one combination of the at least one first transfer function and the at least one second transfer function to produce at least one denoised acoustic data stream.

20. The system of claim 19, wherein the at least one sensor includes at least one radio frequency (RF) interferometer that detects tissue motion associated with human speech production.

21. The system of claim 19, wherein the at least one sensor includes at least one sensor selected from a group consisting of radio frequency devices, electroglottographs, ultrasound devices, acoustic throat microphones, and airflow detectors.

22. The system of claim 19, further comprising:
dividing acoustic data of the at least one acoustic signal into a plurality of subbands;
removing noise from each of the plurality of subbands using the at least one combination of the at least one first transfer function and the at least one second transfer function, wherein a plurality of denoised acoustic data streams are generated; and
combining the plurality of denoised acoustic data streams to generate the at least one denoised acoustic data stream.

23. The system of claim 19, wherein the at least one receiver includes a plurality of independently located microphones.

24. A system for removing noise from acoustic signals, comprising at least one processor coupled among at least one microphone and at least one voicing sensor, wherein the at least one voicing sensor collects physiological data associated with voicing, wherein an absence of voiced information is detected during at least one period using the at least one voicing sensor, wherein at least one noise source signal is received during the at least one period using the at least one microphone, wherein the at least one processor generates at least one transfer function representative of the at least one noise source signal, wherein the at least one microphone receives at least one composite signal comprising acoustic and noise signals, and the at least one processor removes the noise signal from the at least one composite signal using the at least one transfer function to produce at least one denoised acoustic data stream.

25. A signal processing system coupled among at least one user and at least one electronic device, wherein the signal processing system includes at least one denoising subsystem for removing noise from acoustic signals, the denoising subsystem comprising at least one processor coupled among at least one receiver and at least one sensor, wherein the at least one receiver is coupled to receive at least one acoustic signal, wherein the at least one sensor is coupled to receive physiological information associated with human voicing activity, wherein the at least one processor generates a plurality of transfer functions, wherein at least one first transfer function representative of the at least one acoustic signal is generated in response to a determination that voicing information is absent from the at least one acoustic signal for at least one specified period of time, wherein at least one second transfer function representative of the at least one acoustic signal is generated in response to a determination that voicing information is present in the at least one acoustic signal for at least one specified period of time, wherein noise is removed from the at least one acoustic signal using at least one combination of the at least one first transfer function and the at least one second transfer function to produce at least one denoised acoustic data stream.

26. The signal processing system of claim 25, wherein the at least one electronic device includes at least one device selected from a group consisting of cellular telephones, personal digital assistants, portable communication devices, computers, video cameras, digital cameras, and telematics systems.

27. A computer readable medium comprising executable instructions which, when executed in a processing system, remove noise from received acoustic signals by:
receiving at least one acoustic signal;
receiving physiological information associated with human voicing activity;
generating at least one first transfer function representative of the at least one acoustic signal upon determining that voicing information is absent from the at least one acoustic signal for at least one specified period of time;
generating at least one second transfer function representative of the at least one acoustic signal upon determining that voicing information is present in the at least one acoustic signal for at least one specified period of time;
removing noise from the at least one acoustic signal using at least one combination of the at least one first transfer function and the at least one second transfer function to produce at least one denoised acoustic data stream.

28. An electromagnetic medium comprising executable instructions which, when executed in a processing system, remove noise from received acoustic signals by:
receiving at least one acoustic signal;
receiving physiological information associated with human voicing activity;
generating at least one first transfer function representative of the at least one acoustic signal upon determining that voicing information is absent from the at least one acoustic signal for at least one specified period of time;
generating at least one second transfer function representative of the at least one acoustic signal upon determining that voicing information is present in the at least one acoustic signal for at least one specified period of time;
removing noise from the at least one acoustic signal using at least one combination of the at least one first transfer function and the at least one second transfer function to produce at least one denoised acoustic data stream.
